(19) United States
(12) Patent Application Publication
(10) Pub. No.: US 2021/0260147 A1
(43) Pub. Date: Aug. 26, 2021

(54) MITIGATION OF CHRONIC INFANT MALNUTRITION

(71) Applicant: Intermark Partners Strategic Management, LLC, Carmel, IN (US)

(72) Inventor: Glenn H. Sullivan, Carmel, IN (US)

(21) Appl. No.: 17/184,931

(22) Filed: Feb. 25, 2021

Related U.S. Application Data
(60) Provisional application No. 62/981,319, filed on Feb. 25, 2020.

(51) Int. Cl. A61K 36/899; A61P 3/02; A61P 15/14; A23L 33/105; A23L 33/00

(52) U.S. Cl. CPC A61K 36/899; A61P 3/02; A23V 2002/00; A23L 33/105; A23L 33/40; A61P 15/14

(57) ABSTRACT

Disclosed are nutraceutical formulations, and methods of using these nutraceutical formulations for the mitigation of chronic malnutrition in an infant, wherein the infant is breast feeding from a lactating mother ingesting one or more of said nutraceutical formulations in accordance with a prescribed regimen.

### CROSS-REFERENCE TO RELATED APPLICATIONS

The present application claims priority to U.S. Application Ser. No. 62/981,319, filed on Feb. 25, 2020. The entire text of the aforementioned application is incorporated herein by reference in its entirety.

### FEDERALLY SPONSORED RESEARCH OR DEVELOPMENT

[Not Applicable]

### TECHNICAL FIELD

The present invention relates to compositions and methods for the mitigation of chronic infant malnutrition using nutritionally enhanced derivatives (isolate) from Stabilized Rice Bran (SRB) with improved yield characteristics. In certain aspects, the present invention relates to the dietary supplementation of lactating mothers with rice bran enzymatic extracts, for example during the breastfeeding period, with a concomitant increased growth of the breastfed infants and a reduction in anemia prevalence in mothers. In other aspects, the present invention relates to improvements in the quantity and quality of maternal milk.

### BACKGROUND

Chronic infant malnutrition (CIM) is a condition commonly associated with extreme poverty conditions and inadequate caloric-proteic intake by both mothers and infants during pregnancy, lactation and the transition into solid food intake.

CIM still affects about 165 million of children under five years of age in low- and middle-income countries around the world. The consequences of poor nutrition during this critical life stage are permanent intellectual impairment and physiological weakness, constituting in essence a sentence for a life of marginality, underperformance and food welfare.

There exist a need in the art to provide functional foods that provide sustainable nutrition to undernourished populations worldwide. Among other things, the present disclosure provides for the conversion of rice bran to its highest bioactive isolate derivatives in shelf-stable formulations, and for the nutraceutical formulations that mitigate malnutrition, including chronic infant malnutrition. The present disclosure further relates to the dietary supplementation of lactating mothers with rice bran enzymatic extracts (e.g., during the breastfeeding period) with an associated: improvement in the quantity and quality of maternal milk; increase in the growth of the breastfed infants; and a reduction in anemia prevalence in mothers.

### SUMMARY OF THE INVENTION

Provided are nutraceuticals and their use for the mitigation of malnutrition, including chronic infant malnutrition.

In one aspect, the disclosure provides nutraceutical formulations, and methods of using these nutraceutical formulations for the mitigation of chronic infant malnutrition, wherein the infant is breast feeding from a lactating mother ingesting one or more of said nutraceutical formulations in accordance with a prescribed regimen.

In further aspects, the present invention relates to the dietary supplementation of lactating mothers with rice bran enzymatic extracts (e.g., before and/or during the breastfeeding period) with an associated: improvement in the quantity and quality of maternal milk; increase in the growth of the breastfed infants; and a reduction in anemia prevalence in mothers.

### BRIEF DESCRIPTION OF THE DRAWINGS

FIG. 1 shows hematocrit results for blood samples taken at registration of participants (initial) and at the end of the RBEE supplementation window (Final). *Indicates a statistically significant difference at 5%, (p-value=0.0297), according to the student T test for independent samples. Hematocrit values were adjusted by +0.01, taking into consideration sea level altitude.

### DETAILED DESCRIPTION

The Examples that follow are intended to be illustrative of the aspects and embodiments described above. Neither the above disclosure nor the Examples below should be viewed as limiting to the scope of the appended claims. One of skill in the art will appreciate that the disclosure is not limited by the particular terminology which is used to describe and illustrate the various aspects of the disclosure.

To fully capitalize on the nutritional properties of rice bran (RB), a hydrolyzed concentrated rice bran enzymatic extract (RBEE), with significantly increased bioactive capacity, protein, carbohydrate and antioxidant availability has been developed.

A RBEE daily ration was consumed by post-puerperium lactating mothers (LM(s)) for 4.5 months of the exclusive breastfeeding (EBF) period. The nutritional impact on LMs was determined by body mass index (BMI). Anemia prevalence and packed cell volume (PCV) was obtained via hematocrit (HCT). Infant growth was determined monthly following World Health Organization (WHO) guidelines. Infant anthropometric measurements were transformed to growth z scores indicators and analyzed by student t test for independent samples.

Forty grams daily of a RBEE fortified with seven micronutrients, added to enhance the naturally occurring levels of these vitamins and minerals, were added to meet the LM nutritional daily needs. The RBEE daily ration was delivered in 150 ml of potable bottled water and sweetened with 10 g of sugar. The 100% natural, hypoallergenic and gluten-free RBEE is obtained through a patented process by which a unique combination of carbohydrases and proteases, added to stabilized rice bran (SRB) in a multi stage process of centrifugation and dehydration, designed to extract and maximize the bioactive concentration of nutrients from the SRB's lipophilic and hydrophilic fractions. The nutritional composition of the enhanced RBEE supplement is shown in Table 1.

The enhanced RBEE supplement of the present disclosure contains increased amounts of macronutrients and micronutrients (Table 1A), with increased bioavailability resulting from the dual (carbohydrases and proteases) enzymatic process applied to stabilized RB.

As disclosed in greater detail below, the dietary supplementation of lactating mothers with rice bran enzymatic extract during the exclusive breastfeeding period had a significant impact on the mother-infant dyad, with an increased growth of the exclusively breastfed infants and a reduction in anemia prevalence in mothers. For example, anemia prevalence in the LM was reduced from 21.1% to 4%. Additionally, HCT results in LM showed a significant increase (p<0.05) in PCV at the end of the intervention period, from 40.55% to 41.42%. Using WHO metrics, infant growth indicators showed a highly significant (p<0.01) improvement in weight-for-height, weight-for-age and BMI-for-age, and a significant (p<0.05) improvement in head circumference-for-age. Length-for-age group z scores improved from −1.9 to −1.76.

The study population was comprised of post-puerperium LM and their exclusively breastfed infants (EBFI). The sample size (120 participants) was obtained according to the equation for finite populations n=(Nz2pq)/(d2(N−1)+z2pq), where n=sample size; N=600, number of yearly births; pq=0.5 for maximum variability; z=1.96, at 95% confidence level and, d=margin of error, set at 8% (0.08).

The study followed a longitudinal design and participants were enrolled approximately 45 days after birth of the infant. Having the participant LM begin taking the RBEE supplement at this post-partum time allowed them to start their participation at their pre-pregnancy weight. Thus, from the day of enrollment participants consumed the daily ration of the RBEE for a continuous period of 135 days until their infant became 6 months old.

Weight and height measurements, used to determine BMI, were taken from LM at the beginning and the end of the study. Adult weight data was determined using a Tanita digital scale model UM061, while adult height was obtained using a SECA 213 portable stadiometer. In the case of infants, weight was taken using a Tanita pediatric digital scale, model BD 585. The supine length of infants was determined using a SECA 210 infant measuring mat, while the cephalic perimeter was obtained with a SECA head circumference measuring tape. These three growth measurements were taken at registration and repeated at 33-35 day intervals for a period of 135 days and a total of 5 measurements.

Anemia prevalence in LM was determined via hematocrit assay, following WHO micro hematrocrit (HCT) guidelines. (See, Lewis SM, Kumari S. Guidelines on standard operating procedures for haematology. In Guidelines on standard operating procedures for haematology, World Health Organization, New Delhi, India: 2000). Using sterile Marienfeld blood lancets (Lauda-Konigshofen, Germany), a capillary blood sample was collected from the index finger of the non-dominant hand of the LM into sterile 75 mm by 1.1-1.2 mm heparinized Marienfeld capillary tubes. The filled tubes were placed vertically on a holding tray containing sealant clay, to close the bottom end of the tubes. Using a micro HCT analogue centrifuge Gemmy KHT-400, the blood samples were spun for 7 minutes at 12,000 rpm in 24-tube batches for a total of 12 participants per batch, as duplicate samples were obtained for redundancy purposes. Once centrifuged, the packed cell volume (PCV) was determined using a micro hematocrit capillary PCV lineal reader.

In infants, anthropometric measurements were transformed to 5 WHO growth indicators, expressed in z scores, using WHO ANTHRO version 3.2.2. (See, Blossner M, Siyam A, Borghi E, Onyango A, & Onis M. World Health Organization. Software for assessing growth and development of the world's children. Geneva: WHO; 2011.) Growth indicators included weight-for-length (WHZ), weight-for-age (WAZ), length-for-age (HAZ), head circumference-for-age (HCZ) and body mass index-for-age (BAZ). Using Infostat version 2013, a Student t test for independent samples was run on the 5 growth z scores. Prevalence of chronic and acute malnutrition was determined prior to and at the end of the consumption window.

In LM, weight and height data was used to determine BMI prior and at the end of the RBEE supplementation period and classified according to WHO guidelines. A Student t test for independent samples was performed on the LM BMI data obtained prior to and after the consumption window. Prevalence of anemia was also determined at the outset and at the completion of the field trials, applying a 37% PCV cutoff after a +1 correction factor for altitude. A student t test was used to detect any significant variation in the HCT values at the beginning and the end of the supplementation window.

The general information and initial anthropometric measurements of both LM and EBFI is documented in Table 2. Of an initial 153 infants, 83 were female and 70 were male with an average age of 46 days. The age of the infants at registration ranged between 37 and 67 days old. The average age of participating LM was 26.9 years, with the oldest participant being 42 years of age. The initial HCT (corrected +1 for altitude) in LM averaged 40.55%, with a minimum value of 28% PCV and maximum of 51% PCV. The initial BMI in LM was equal to 22.5. The growth data taken among infants at registration showed that the average initial WHZ score was in the normal range at 0.17, while mean WAZ and HCZ scored below normal at −1.6 and −1.3, respectively. Average HAZ was calculated at −1.9, very close to the CM −2 cutoff point.

### Growth of EBFI During LM Supplementation

The average weight gain for infants during the trial was 3 kg, equivalent to a monthly gain of 677 g (1.49 lb). As for length, infants exhibited a growth of 10.45 cm at a rate of 2.36 cm per month, while their head circumference grew at a monthly rate of 1.14 cm, for a total of 5.11 cm in the 135 day period (Table 3).

As disclosed in Table 4, a number of infants were categorized as malnourished at the time of registration in the study. Slightly over 7% fell in either the severe (SAM) or moderate (MAM) acute malnutrition categories. At the average age of 46 days old, 43% of the infants were chronically malnourished (HAZ≤−2), 18.3% of them severely. An important number of infants also exhibited retardation in cranial growth, with 25% of them exhibiting HCZ values ≤−2.

By the end of day 135 the overall nutritional indicators had improved. As shown in Table 4, no infants were acutely malnourished by the end of the study and only 6.2% remained in the “at risk” category, a similar prevalence to that found in BAZ. HAZ showed a slight improvement overall, decreasing the chronic malnutrition (CM) prevalence from 43% to 39%. However, the severe CM prevalence was reduced by 45%, affecting 18.3% of the infants at registration, compared to 10% by the end of the nutritional supplementation window. HCZ scores found that over 25% of the infants exhibited an initial delay in cranial growth, 7.2% of them severely. By the end of the field study, moderate to severe cranial growth delay had fallen to 11.6% of the infants, with only 1.5% of them in the severely undersized category according to HCZ.

A summary of descriptive statistics for the 5 tested growth indicators is shown in Table 5. Based on a percentile scale, an improvement of 41.9 percentile points for BAZ was obtained at the end of the study. Both WHZ and WAZ showed similar gains with 13.44 and 16.29 percentile points, respectively. HCZ exhibited a 5.71 percentile points increase, ending at the 15.39 percentile mark, while HAZ increased 1.05 points.

Statistical analysis determined that the most significant growth occurred during the first half of the trial, between days 0 and 67 of the supplementation (Table 6). The Student t test for independent samples showed highly significant gains (p<0.01) for WHZ, WAZ and BAZ during the first half of the daily supplementation, but no statistical significant differences were found in their rate of growth during the second half of the trial, between days 67 and 135. No significant improvement in the rate of growth was found in the HAZ at any stage of the trail. In contrast, the growth indicator for head circumference, HCZ, showed a significant (p<0.05) improvement in the z scores obtained at registration (0 days) and at the end of the intervention period (135 days).

### Nutritional Status of Lactating Mothers

### Body Mass Index

The average BMI at registration for LM was 22.5 kg/m² (±2.6). Of the 4.6% underweight LM before supplementation, all the cases fell under the mild thinness category with BMIs between 17 and 18.49 kg/m² (Table 7). Slightly over 80% were in a normal BMI range while 15% were overweight with only one obesity case. By the end of the field trials the prevalence of underweight LM had increased to almost 11%, 13 of them exhibiting mild thinness and one moderate thinness. Sixty nine percent of the LM fell in the normal category while 26 of 129 LM were overweight, equivalent to 20.2% of the participants. The average BMI by the end of the intervention was 22.8 (±3.2) kg/m².

### Anemia Prevalence (Hematocrit)

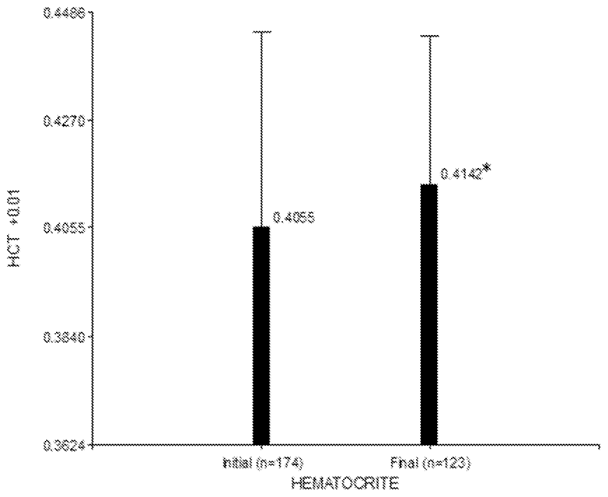

The average HCT results, as disclosed in FIG. 1, were equal to 40.55% PCV before taking the RBEE supplement. After the 135 day supplementation period the average HCT value had increased to 41.42% a statistically (p<0.05) significant increase, according to the Student t test for independent samples. In addition, the standard deviation had decreased from 3.9% to 2.9% showing a tighter grouping of HCT results.

According to the initial HCT results, 12.1% of LM (n=174) were categorized as anemic with a <37% HCT result. By the end of the supplementation period the prevalence of anemia had been reduced to 4% (n=123). If the borderline results were included (HCT≤37), the initial prevalence of anemia increased to 21.2% of the registered LM, decreasing to 11.4% by the end of the nutritional intervention.

### SUMMARY

A significant improvement (p<0.05) was found in the average HCT among LM by the end of the nutritional intervention period. Along with this improvement in HCT a threefold reduction in the prevalence of anemia was achieved by the end of the study. There was also a 6.3% increase in the underweight category among LM by the end of the study, more than doubling the initial underweight prevalence.

The length (average 52.3 cm) of participating EBFI at the very young age of 46 days old shows that 43% exhibited moderate (24.8%) or severe (18.3%) growth retardation. Length at birth is an important anthropometric indicator with known negative consequences in the future individual growth and can be affected by a retardation in intrauterine growth due to maternal weight gain or deficient BMI during pregnancy.

There was an improvement in the average length of infants, particularly among the severely affected by CM. A breakdown of the chronic undernutrition categories of EBFI at the beginning of the study showed that 18.3% and 24.8% were afflicted by severe and moderate chronic undernutrition, respectively. By the end of the study the prevalence of severe chronic undernutrition had fallen to 10.1% of the participants, a 45% reduction. The moderate malnourished prevalence increased from 24.8% initially to 29.5% by the end of the field study which can be explained by a probable shift of infants from the severe to the moderate undernutrition category by the end of the intervention period.

According to WHO growth charts for infants between 0 and 6 months of age, both boys and girls grow an average of 11 cm in the 1.5 months to 6 months period. In the case of the RBEE supplementation reported in this paper, infants grew an average of 10.45 cm, a length growth very close to the expected normal increase. In the first half of the study, between 1.5 (46 days) and 3.7 (113 days) months old infants grew an average of 6.41 cm while in the second 67 days of the supplementation window (ages 3.7 to 6 months old) infants grew 4.04 cm, a growth rate very close to the normal rate of 4.5 cm.

At the end of the study, head circumference growth delay was reduced by 55%, from the initial 25.5% to 11.6% prevalence. Furthermore, only 1.5% of the participants showed a severe delay in cephalic perimeter growth, an 80% reduction from the initial 7.2% prevalence of severe (HCZ≤−3) head growth retardation at the beginning of the study. Student's t test showed a significant (p<0.05) growth in peripheral head size of EBFI in the 135-day long study.

WAZ and BAZ scores improved among participating infants at the end of the study. Thirty four percent of infants were found to be moderately or severely compromised at the beginning of the intervention period according to WAZ, but this prevalence was reduced to 11% by the study's end. At registration, 18% of the infants showed BAZ scores ≤−2, with no compromised infants at the end of the field stage.

In the three growth indicators in which a highly significant (p<0.01) improvement was detected (WHZ, WAZ, BAZ), a significant growth boost took place in the first half (0-67 days) of the study (Table 6). Further analysis of the data shows that in the first half of the nutritional window infants gained an average of 1.93 kg almost twice the weight the infants gained (1.07 kg) during the second half of the field study. The overall average weight gain (3.0 kg) during the 135 days of the nutritional window was better than the expected normal weight gain shown in WHO 0-6 months growth charts, equivalent to 2.6 kg and 2.8 kg for girls and boys, respectively.

The vast majority of the LMs experienced increased milk production starting in the 15-20 day range after having initiated the supplementation. Increased milk quantity allowed for more frequent and longer breastfeeding periods and was important factor in the improvement observed in their infants.

The dietary supplementation of breastfeeding mothers with RBEE during the 46 to 180 days post-partum period has a significant positive impact on the mother-infant dyad, as the apparent increase in breast milk production favored an increased growth of their exclusively breastfed infants. In addition, anemia prevalence was significantly reduced among mothers taking the daily supplement. Formulating infant nutrition supplements with RBEE, to be consumed by the lactating mother during the EBF mitigates chronic or acute malnutrition in nutritionally deficient populations.

TABLE 1
Nutritional profile of enhanced rice bran enzymatic extract for lactating women.

| | Rice bran enzymatic | | Per ration (40 g) | | |
|---|---|---|---|---|---|
| | extract typical analysis | | | | % Lactating |
| Nutrient | 100 g | 40 g | Added | Total | women RDA¹ |
| Energy (calories) | 448-468 | 180-188 | | 180-188 | 7.0 |
| Protein (g) | 12-15 | 4.8-6.0 | | 4.8-6.0 | 5.0 |
| Fat (g) | 27.90 | 11.16 | | 11.16 | 12.6 |
| Carbohydrates (g) | 58.40 | 23.36 | | 23.36 | 6.4 |
| Fiber (g) | 3.00 | 1.20 | | 1.20 | |
| Vitamin A (mcg) | 106.70 | 42.68 | 400.00 | 442.68 | 44.3 |
| Thiamin (mg) | 3.14 | 1.26 | | 1.26 | 96.6 |
| Riboflavin (mg) | 2.84 | 1.14 | | 1.14 | 71.0 |
| Niacin (mg) | 7.26 | 2.90 | | 2.90 | 17.1 |
| Pantothenic acid (mg) | 6.29 | 2.52 | | 2.52 | 35.9 |
| Pyridoxin(mg) | 3.51 | 1.40 | | 1.40 | 70.2 |
| Biotin (mcg) | 5.25 | 2.10 | | 2.10 | 6.0 |
| Folate (mcg) | 126.81 | 50.72 | 487.00 | 537.72 | 100.0 |
| Vitamin B12 (mcg) | 0.64 | 0.26 | 2.80 | 3.06 | 100.0 |
| Vitamin C (mg) | 10.62 | 4.25 | 100.00 | 104.25 | 100.0 |
| Vitamin D (mcg) | 1.91 | 0.76 | 2.50 | 3.26 | 65.3 |
| Vitamin E (mg ET) | 1.56 | 0.62 | | 0.62 | 3.3 |
| Tocophenols (mg) | 2.11 | 0.84 | | 0.84 | |
| Tocotrienols (mg) | 2.28 | 0.91 | | 0.91 | |
| Phytosterols (mg) | 939.88 | 375.95 | | 375.95 | |
| Gamma-oryzanol (mg) | 250.34 | 100.14 | | 100.14 | |
| Ferulic acid (mcg) | 3502.50 | 1401.00 | | 1401.00 | |
| Vitamin K (mcg) | 8.08 | 3.23 | | 3.23 | 5.6 |
| Calcium (mg) | 145.70 | 58.28 | | 58.28 | 5.8 |
| Chromium (mcg) | 283.91 | 113.56 | | 113.56 | 252.0 |
| Copper (mcg) | 937.39 | 374.96 | | 374.96 | 72.0 |
| Iron (mg) | 4.14 | 1.66 | 7.39 | 9.05 | 58.0 |
| Magnesium (mg) | 542.30 | 216.92 | | 216.92 | 79.0 |
| Manganese (mg) | 12.95 | 5.18 | | 5.18 | 2.6 |
| Molybdenum (mcg) | 35.85 | 14.34 | | 14.34 | 28.7 |
| Phosphorus (mg) | 1366.00 | 546.40 | | 546.40 | 78.1 |
| Potassium (g) | 1.60 | 0.64 | | 0.64 | 12.5 |
| Zinc (mg) | 5.00 | 2.00 | 11.70 | 13.70 | 60.5 |

¹Required daily allowance as recommended by the Nutrition Institute for Central America and Panamá, INCAP.

TABLE 1A
Comparison of fat and protein content between stabilized rice bran
subjected to either standard or enhanced enzymatic treatment.

| | Average ± standard deviation of 4 samples | | | | | | | |
|---|---|---|---|---|---|---|---|---|
| | Begin | Decant | | Dry basis | | | % As-Is | As-Is |
| Isolate | weight (g) | weight (g) | % Solids | weight (g) | % As-Is Fat | As-Is fat (g) | Protein | Protein(g) |
| Standard | 180 | 103.75 ± | 7.14 ± | 7.39 ± | 0.93 ± | 0.95 ± | 0.83 ± | 0.85 ± |
| RBEE | | 5.93 | 0.24 | 0.23 | 0.27 | 0.24 | 0.06 | 0.1 |
| Enhanced | 180 | 113 ± | 11.4 ± | 12.9 ± | 2.5 ± | 2.82 ± | 2.65 ± | 3 ± |
| RBEE | | 2.45 | 0.19 | 0.25 | 0.08 | 0.15 | 0.1 | 0.18 |
| Increased | 0 | 9.25 | 4.27 | 5.5 | 1.56 | 1.87 | 1.82 | 2.14 |
| amount (g) | | | | | | | | |
| % Increase | 0.0% | 89.0% | 59.8% | 74.4% | 167.6% | 195.6% | 220.9% | 248.8% |

TABLE 2
General information and nutritional/growth
data of participants at registration.

| | | Exclusively |
|---|---|---|
| | Lactating | breastfed |
| Age/Anthropometry/ | mothers | infants |
| Growth z scores | (n = 153) | (n = 153) |
| Age | | |
| years | 26.90 ± 6.6 | |
| days | | 46 ± 0.04 |
| Initial Hematocrit | 40.55 ± 4 | |
| Initial BMI | 22.50 ± 2.6 | |
| Anthropometry | | |
| weight (kg) | 48.70 ± 6.4 | 3.98 ± 0.71 |
| height (m) | 1.47 ± 0.05 | |
| length (cm) | | 52.3 ± 2.43 |
| head circumference | | 36.3 ± 1.41 |
| (cm) | | |
| Growth Indicator | | |
| (z scores) | | |
| weight for length | | 0.17 ± 1.3 |
| weight for age | | −1.6 ± 1.3 |
| length for age | | −1.9 ± 1.21 |
| head circum. for | | −1.3 ± 1.14 |
| age | | |
| body mass index | | −0.82 ± 1.33 |
| for age | | |

TABLE 3
Mean anthropometrics measurements of EBF infants
during LM daily supplementation with RBEE

| | | Average | | |
|---|---|---|---|---|
| | Days into LM | | | head circumference |
| Measurement | supplementation | weight (kg) | length (cm) | (cm) |
| 0 | 0 | 3.98 ± 0.71 | 52.31 ± 2.44 | 36.33 ± 1.41 |
| 1 | 30 | 4.97 ± 0.76 | 55.87 ± 2.17 | 38.05 ± 1.35 |
| 2 | 67 | 5.91 ± 0.79 | 58.72 ± 2.28 | 39.44 ± 1.25 |
| 3 | 97 | 6.47 ± 0.8 | 60.78 ± 2.25 | 40.48 ± 1.37 |
| 4 | 135 | 6.98 ± 0.87 | 62.76 ± 2.27 | 41.44 ± 1.31 |

TABLE 4
Percentage (%) of EBFI (n = 153) in each z score category
according to the initial and final nutritional evaluations

| | z score (%) | | | | | | | |
|---|---|---|---|---|---|---|---|---|
| | | −2.99 | −1.99 | −0.99 | 0.01 | 1 & | 2 & | |
| Growth indicator | ≤−3 | & −2 | & −1 | & 0 | & 0.99 | 1.99 | 2.99 | ≥3 |
| weight-for-length (WHZ) | | | | | | | | |
| Initial | 1.3 | 5.9 | 9.8 | 24.8 | 32.7 | 18.3 | 7.2 | |
| Final | | | 6.2 | 26.3 | 33.3 | 27.2 | 6.2 | 0.8 |
| weight-for-age (WAZ) | | | | | | | | |
| initial | 17.1 | 17.1 | 26.1 | 30.5 | 8.5 | 0.7 | | |
| Final | 0.8 | 10.1 | 34.1 | 29.4 | 20.2 | 5.4 | | |
| length-for-age (HAZ) | | | | | | | | |
| Initial | 18.3 | 24.8 | 35.3 | 16.3 | 4.6 | 0.7 | | |
| Final | 10.1 | 29.5 | 38.7 | 18.6 | 3.1 | | | |
| head | | | | | | | | |
| circum.-for-age (HCZ) | | | | | | | | |
| Initial | 7.2 | 18.3 | 32.1 | 31.3 | 10.4 | 0.7 | | |
| Final | 1.5 | 10.1 | 41.8 | 32.6 | 11.7 | 2.3 | | |
| BMI-for-age (BAZ) | | | | | | | | |
| Initial | 9.2 | 9.2 | 22.2 | 28.8 | 23.5 | | | |
| Final | | | 8.5 | 34.2 | 31.1 | 21.7 | 3.7 | 0.8 |

TABLE 5
Summary of descriptive statistics for EBFI growth
indicators at 0, 67 and 135 days into the daily
nutritional supplementation of lactating mothers.

| Anthropometric | Growth | z score | | |
|---|---|---|---|---|
| measurement | indicator | Mean ± sd | Median | Percentile |
| 0 (0 days) | WHZ | 0.17 ± 1.3 | 0.32 | 56.75 |
| 2 (67 days) | WHZ | 0.63 ± 1.11 | 0.68 | 73.53 |
| 4 (135 days) | WHZ | 0.53 ± 0.99 | 0.46 | 70.19 |
| 0 (0 days) | WAZ | −1.6 ± 1.3 | −1.39 | 5.48 |
| 2 (67 days) | WAZ | −0.94 ± 1.19 | −0.89 | 17.36 |
| 4 (135 days) | WAZ | −0.78 ± 1.04 | −0.9 | 21.77 |
| 0 (0 days) | HAZ | −1.9 ± 1.21 | −1.85 | 2.87 |
| 2 (67 days) | HAZ | −1.72 ± 1.25 | −1.72 | 4.27 |
| 4 (135 days) | HAZ | −1.76 ± 1 | −1.72 | 3.92 |
| 0 (0 days) | HCZ | −1.3 ± 1.14 | −1.16 | 9.68 |
| 2 (67 days) | HCZ | −1.05 ± 1.18 | −1.14 | 14.69 |
| 4 (135 days) | HCZ | −1.02 ± 0.91 | −1.11 | 15.39 |
| 0 (0 days) | BAZ | −0.82 ± 1.33 | −0.63 | 20.61 |
| 2 (67 days) | BAZ | 0.14 ± 1.14 | 0.13 | 55.57 |
| 4 (135 days) | BAZ | 0.32 ± 1.01 | 0.19 | 62.55 |

TABLE 6
Student T test results for z scores of 5 growth indicators obtained
at the 0, 67 and 135 days into the LM daily RBEE supplementation

| | Statistical | | | | | | | | |
|---|---|---|---|---|---|---|---|---|---|
| Growth | group | | n | | mean | | mean 2 - | | |
| Indicator | Group 1 | Group 2 | Group 1 | Group 2 | Group 1 | Group 2 | mean 1 | T | p-value |
| WLZ | 0 | 67 | 153 | 128 | 0.17 | 0.63 | 0.46 | −3.15 | 0.002 |
| WLZ | 0 | 135 | 153 | 129 | 0.17 | 0.53 | 0.36 | −2.64 | 0.009 |
| WLZ | 67 | 135 | 128 | 129 | 0.63 | 0.53 | −0.1 | 0.75 | 0.455 |
| WAZ | 0 | 67 | 153 | 128 | −1.6 | −0.94 | 0.66 | −4.41 | <0.0001 |
| WAZ | 0 | 135 | 153 | 129 | −1.6 | −0.78 | 0.81 | −5.85 | <0.0001 |
| WAZ | 67 | 135 | 128 | 129 | −0.94 | −0.78 | 0.15 | −1.1 | 0.272 |
| HAZ | 0 | 67 | 153 | 128 | −1.9 | −1.72 | 0.18 | −1.25 | 0.213 |
| HAZ | 0 | 135 | 153 | 129 | −1.9 | −1.76 | 0.14 | −1.05 | 0.293 |
| HAZ | 67 | 135 | 128 | 129 | −1.72 | −1.76 | −0.04 | 0.32 | 0.752 |
| HCZ | 0 | 67 | 153 | 128 | −1.3 | −1.05 | 0.24 | −1.73 | 0.084 |
| HCZ | 0 | 135 | 153 | 129 | −1.3 | −1.02 | 0.28 | −2.26 | 0.024 |
| HCZ | 67 | 135 | 128 | 129 | −1.05 | −1.02 | 0.03 | −0.26 | 0.798 |
| BAZ | 0 | 67 | 153 | 128 | −0.82 | 0.14 | 0.96 | −6.44 | <0.0001 |
| BAZ | 0 | 135 | 153 | 129 | −0.82 | 0.32 | 1.13 | −8.12 | <0.0001 |
| BAZ | 67 | 135 | 128 | 129 | 0.14 | 0.32 | 0.17 | −1.29 | 0.199 |

TABLE 7
Body mass index classification of women (according to WHO metrics) prior to
and at finalization of the 135 day RBEE nutritional supplementation window

| | | Lactating mothers | | | |
|---|---|---|---|---|---|
| | BMI (kg/m²) | Initial number | | Final number | |
| Classification | Main cut-off points | (n = 153) | % | (n = 129) | % |
| Underweight | <18.50 | 7 | 4.6% | 14 | 10.9% |
| Severe thinness | <16.00 | 0 | | 0 | |
| Moderate thinness | 16.00-16.99 | 0 | | 1 | |
| Mild thinness | 17.00-18.49 | 7 | | 13 | |
| Normal range | 18.50-24.99 | 123 | 80.4% | 89 | 69.0% |
| Overweight | ≥25.00 | 23 | 15.0% | 26 | 20.2% |
| Pre-obese | 25.00-29.99 | 22 | | 21 | |
| Obese | ≥30.00 | 1 | | | |
| Obese class I | 30.00-34.99 | | | 5 | |
| Obese class II | 35.00-39.99 | | | | |
| Obese class III | ≥40.00 | | | | |

1. A method for the dietary supplementation of lactating mother, said method comprising administering to said mother a rice bran enzymatic extract in an amount effective to reduce anemia in said mother, and simultaneously increase the growth of an infant breast feeding from said mother.
